(19) United States
(12) Patent Application Publication
(10) Pub. No.: US 2014/0276790 A1
(43) Pub. Date: Sep. 18, 2014

(54) DEVICES FOR TISSUE SEPARATION AND RELATED METHODS OF USE

(71) Applicant: Boston Scientific Scimed, Inc., Maple Grove, MN (US)

(72) Inventors: Samuel RAYBIN, Marlborough, MA (US); Paul SMITH, Smithfield, RI (US); Gary S. KAPPEL, Acton, MA (US); Naroun SUON, Lawrence, MA (US)

(73) Assignee: Boston Scientific Scimed, Inc., Maple Grove, MN (US)

(21) Appl. No.: 14/206,353

(22) Filed: Mar. 12, 2014

Related U.S. Application Data
(60) Provisional application No. 61/780,133, filed on Mar. 13, 2013.

(51) Int. Cl. A61B 18/14

(52) U.S. Cl. CPC A61B 18/1492
USPC 606/41

(57) ABSTRACT

The present disclosure is directed to a medical instrument. The medical instrument may include a shaft having a distal end configured to cauterize tissue. An expandable member may be positioned adjacent the distal end of the shaft. The expandable member may be configured for deployment between a collapsed configuration and an expanded configuration.

### CROSS-REFERENCE TO RELATED APPLICATIONS

This application claims the benefit of priority from U.S. Provisional Application No. 61/780,133, filed on Mar. 13, 2013, the entirety of which is incorporated by reference herein.

### DESCRIPTION OF THE EMBODIMENTS

1. Technical Field

Embodiments of the present disclosure relate generally to medical instruments. More particularly, embodiments of the disclosure relate to medical instruments for use in medical applications, such as, for example, resection and dissection procedures. Embodiments of the disclosure also cover methods of using such instruments.

2. Background of the Disclosure

Organ walls are composed of several layers: the mucosa (the surface layer), the submucosa, the muscularis (muscle layer), and the serosa (connective tissue layer). In gastrointestinal, colonic, and esophageal cancer, lesions or cancerous masses may form along the mucosa and often extend into the lumens of the organs. Conventionally, the condition is treated by cutting out a portion of the affected organ wall. This procedure, however, may cause discomfort to patients, and pose health risks.

Physicians have adopted minimally invasive techniques called endoscopic mucosal resection (EMR) and endoscopic submucosal dissection (ESD). EMR methods are typically used for removal of small cancerous or abnormal tissues (i.e., polyps), and ESD methods are typically used for en bloc removal of large cancerous or abnormal tissues (e.g., lesions). These procedures are generally performed with an endoscope, which is a long, narrow elongated member optionally equipped with a light, imaging equipment, and other instruments. During these procedures, the endoscope may be passed through a percutaneous incision, passed down the throat, or guided through the rectum to reach tissue targeted for resection or dissection, such a tissue having an abnormality such as a lesion or cancerous mass in an affected organ. The lesion is generally identified and marked. The mucosal layer containing the lesion is then separated from the underlying tissue layers using a medical instrument extending through a working channel of the endoscope. The lesion is subsequently removed using the same or different medical instrument. Conventionally, tissue is removed by employing a cutting device such as a wire loop, which may be adapted for electrocautery. Subsequently, excised tissue may be extracted for examination or disposal.

These procedures may suffer from long procedure times, perforation risks, insufficient area removal capabilities, and seeding risk from leaving sections of cancerous tissue behind. As such, there exists a need for improved medical instruments and procedures that effectively resect and/or dissect a targeted tissue without damaging the surrounding tissue or muscle layers of the organ, and that allow for more complete, efficient removal of larger areas of tissue.

### SUMMARY OF THE DISCLOSURE

Embodiments of the present disclosure provide devices and methods for effectively separating tissue targeted for resection and/or dissection from any underlying tissue layers using a minimally invasive surgical system.

One embodiment of the present disclosure is directed to a medical instrument. The medical instrument may include a shaft having a distal end configured to cauterize tissue. The medical instrument may also include an expandable member positioned adjacent the distal end of the shaft. The expandable member may be configured for deployment between a collapsed configuration and an expanded configuration. At least a portion of the expandable member may have a wedge-shaped profile in the expanded configuration.

In various embodiments, the medical instrument may include one or more of the following additional features: wherein the expandable member has a substantially flat base in the expanded configuration; wherein the shaft has a cauterizing tip; wherein the expandable member includes a substantially flat base and a body having a substantially flat top surface, wherein the substantially flat top surface and the substantially flat base meet at an edge that is straight and oriented perpendicular to a longitudinal axis of the shaft; further including a channel extending through the shaft to a distal opening, wherein the channel is configured to deliver fluid; wherein the expandable member is a balloon.

Another embodiment of the present disclosure is directed to a medical instrument. The medical instrument may include a shaft having a distal end configured to cauterize tissue. The medical instrument may also include an expandable member configured for insertion between a first tissue layer and a second tissue layer adjacent the first tissue layer. The expandable member may be configured to expand from a collapsed state to an expanded state to separate the first tissue layer from the second tissue layer. At least a portion of the expandable member may have a substantially flat base in the expanded state.

In various embodiments, the medical instrument may include one or more of the following additional features: wherein a body of the expandable member has a substantially tapered shape in the expanded state; wherein at least a portion of the expandable member has a wedge-shaped profile in the expanded state; wherein the shaft has a cautery tip; wherein the expandable member includes a plurality of inflation chambers; wherein the expandable member includes a substantially flat base and a body having a substantially flat top surface, wherein the substantially flat top surface and the substantially flat base meet at an edge that is straight and oriented perpendicular to a longitudinal axis of the shaft; further including a channel extending through the shaft to a distal opening, wherein the channel is configured to deliver fluid; and wherein the expandable member is a balloon.

Another embodiment of the present disclosure is directed to a method of separating tissue layer. The method may include positioning a distal portion of a medical instrument, including a shaft having a distal end configured to cauterize tissue, and an expandable member adjacent the distal end of the shaft, adjacent a tissue site. The method may also include inserting the distal portion between a first tissue layer and a second tissue layer at the tissue site. The method may also include expanding the expandable member from a collapsed configuration to an expanded configuration to separate the first tissue layer from the second tissue layer. At least a portion of the expandable member may have a wedge-shaped profile in the expanded configuration.

In various embodiments, the method may include one or more of the following additional features: further including collapsing the expandable member; positioning the expandable member at a second location adjacent the tissue site; and expanding the expandable member to separate the first tissue layer from the second tissue layer; further including, prior to expanding the expandable member, injecting fluid between the first tissue layer and the second tissue layer; further including, after expanding the expandable member, inserting a treatment device between the first tissue layer and the second tissue layer to dissect a portion of the first tissue layer; wherein the first tissue layer is the mucosal layer, and the second tissue layer is the muscularis layer; and further including, prior to inserting the distal portion between the first tissue layer and the second tissue layer, cutting the first tissue layer with the distal end of the instrument.

In accordance with another embodiment of the present disclosure, a tool may include a cap member having a proximal end and a distal end. The proximal end may be configured to be secured to a distal portion of an introduction sheath. Further, at least one of the cap member or a portion of the cap member may be transparent. A tip may extend distally from the distal end of the cap member, and an expandable member may be secured to the cap member. In addition, the expandable member may be configured to transition between a collapsed state and an expanded state.

In various embodiments, the tool may include one or more of the following additional features: the tip may be configured to cauterize tissue; the tip may include an atraumatic configuration; the expandable member may be at least one of an expandable balloon or an expandable basket; the tool may further include a visualization mechanism; the expandable member or a portion of the expandable member may be transparent; the at least one expandable member may include a plurality of expandable members; the tip may be configured to move independently of the cap member; a reciprocation mechanism for longitudinally reciprocating the cap member; the reciprocation mechanism may extend through the introduction sheath and operatively connect to the cap member, and drive reciprocation of the cap member longitudinally relative to the introduction sheath; when in the expanded configuration, the at least one expandable member may be configured to extend radially away from an axis of the cap member; the tip may be configured to move independently of the cap member; and a portion of the cap member may be configured for reciprocal movement relative to a remainder of the cap member.

According to another embodiment, an endoscopic tool may include a cap member having a proximal end and a distal end, wherein the proximal end is configured to be secured to a distal portion of an introduction sheath having a proximal end, a distal end, and a lumen extending therebetween. In addition, the entire cap member or a portion of the cap member may be transparent, and a tip configured to cut tissue, may extend distally from the distal end of the cap member. The tip may be configured to move independently of the cap member. An expandable member may be secured to the cap member, wherein the expandable member may be configured to transition between a collapsed state and an expanded state.

In various embodiments, the endoscopic tool may further include one or more of the following additional features: the at least one expandable member may include a plurality of expandable members; the tip may include an atraumatic configuration; the tip may include a cautery element; the expandable member may be one of an expandable balloon or an expandable basket; the endoscopic tool may further including a visualization mechanism; and a portion of the cap member may be configured for reciprocal movement relative to a remainder of the cap.

A further aspect of the present disclosure includes a method for dissecting tissue. The method may include introducing an endoscopic tool into a body cavity. The endoscopic tool may include a cap member having a proximal end and a distal end. At least one of the cap member or a portion of the cap member may be transparent. The endoscopic tool may further include a tip extending distally from the distal end of the cap member, and at least one expandable member secured to the cap member, wherein the expandable member may be configured to transition between a collapsed state and an expanded state. The method may further include viewing the surrounding body cavity through the cap member, and expanding the expandable member to separate adjacent tissue layers.

In various embodiments, the method may include one or more of the following: the at least one expandable member may include a plurality of expandable members; and piercing a tissue layer of the adjacent tissue layers with the tip of the endoscopic tool.

Additional objects and advantages of the disclosure will be set forth in part in the description which follows, and in part will be obvious from the description, or may be learned by practice of the disclosure. The objects and advantages of the disclosure will be realized and attained by means of the elements and combinations particularly pointed out in the appended claims.

It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate embodiments of the disclosure and together with the description, serve to explain the principles of the disclosure.

FIG. 1 illustrates an exemplary endoscope for use with a medical instrument having a distal portion including a distal tool and an expandable member, according to a first embodiment of the disclosure;

FIG. 2 is a partial perspective view of the distal portion of the medical instrument with the expandable member in a collapsed configuration, according to a first exemplary embodiment of the disclosure;

FIG. 3 is a partial perspective view of the distal portion of the medical instrument with the expandable member in an expanded configuration, according to a first exemplary embodiment of the disclosure;

FIG. 4 is a cross-sectional view of the distal portion of the medical instrument shown in FIG. 3, according to a first exemplary embodiment of the disclosure;

FIG. 5A illustrates the distal tool forming a space A between a first tissue layer and a second tissue layer at a tissue site, according to a first exemplary embodiment of the disclosure;

FIG. 5B illustrates the expandable member expanding from the collapsed configuration to the expanded configuration to create an area B separating the first tissue layer from the second tissue layer at the tissue site, according to a first exemplary embodiment of the disclosure;

FIG. 6 is a partial perspective view of a distal portion of a medical instrument with a distal tool and an expandable member, the expandable member being in a collapsed configuration, according to a second exemplary embodiment of the disclosure;

FIG. 7 is a partial perspective view of the distal portion of the medical instrument with the expandable member in an expanded configuration, according to a second exemplary embodiment of the disclosure;

FIG. 8A illustrates a distal tool forming a space A between a first tissue layer and a second tissue layer at a tissue site, according to second exemplary embodiment of the disclosure;

FIG. 8B illustrates a fluid being injected in the space A, according to a second exemplary embodiment of the disclosure;

FIG. 8C illustrates the expandable member expanding from the collapsed configuration to the expanded configuration to create an area B separating the first tissue layer from the second tissue layer at the tissue site, according to a second exemplary embodiment of the disclosure;

FIG. 9 illustrates an alternative embodiment of the expandable member, according to an exemplary embodiment of the disclosure;

FIG. 10 illustrates another alternative embodiment of the expandable member, according to an exemplary embodiment of the disclosure;

FIGS. 11A and 11B illustrate another exemplary medical device in collapsed and expanded states, respectively, according to an embodiment of the present disclosure;

FIG. 12 is a side view of an alternative embodiment of a medical device, in accordance with the principles disclosed herein;

FIG. 13 is a side view of another alternative embodiment of a medical device, in accordance with the principles disclosed herein; and

FIGS. 14A and 14B illustrate an exemplary method of using medical devices disclosed herein.

### DETAILED DESCRIPTION OF THE EMBODIMENTS

Reference will now be made in detail to exemplary embodiments of the disclosure, examples of which are illustrated in the accompanying drawings. Wherever possible, the same reference numbers will be used throughout the drawings to refer to the same or like parts.

Embodiments of the present disclosure relate to systems and methods for separating target tissue from any underlying tissue layers. For example, the device may separate tissue layers from the mucosal walls of the colon, esophagus, stomach, or duodenum facilitating the later removal of undesired tissue.

FIG. 1 depicts an endoscope 10 according to an exemplary embodiment of the disclosure. Endoscope 10 may be used for procedures within or adjacent to various body organs, such as, an esophagus, a heart, a stomach, a pelvic area, a bladder, an intestine, or any other portion of a gastrointestinal, urinary, or pulmonary tract. Endoscope 10 may be configured for insertion into a patient's body through an anatomical opening. In some embodiments, endoscope 10 may be used in natural orifice transluminal endoscopic surgery (NOTES) procedures or single incision laparoscopic surgical (SILS) procedures. Accordingly, endoscope 10 may be shaped and sized for placement into a patient via a body cavity or an incision.

Endoscope 10 includes a proximal end 10a, a distal end 10b, and an outer tube 12 extending between proximal end 10a and distal end 10b. For purposes of this disclosure, “proximal” refers to the end closer to the device operator during use, and “distal” refers to the end further from the device operator during use.

A handle portion 14 is disposed at proximal end 10a of endoscope 10. Handle portion 14 may be any known, suitable handle. As illustrated in FIG. 1, handle portion 14 includes rotatable control knobs 16, which may be connected to control wires or cables (not shown) within outer tube 12, to provide up/down and left/right steering of distal end 10b of endoscope 10. Handle portion 14 additionally includes an adapter 18 for allowing delivery of electrical energy, signals, and/or light to distal end 10b of endoscope 10.

Outer tube 12 extends distally from handle portion 14 and terminates at a distal end 12b. Outer tube 12 may be a flexible tube, made from any suitable biocompatible material known to one of ordinary skill in the art and having sufficient flexibility to traverse tortuous anatomy. Such materials may include, but are not limited to, rubber, silicon, synthetic plastic, stainless steel, metal-polymer composites, and metal alloys of nickel, titanium, copper cobalt, vanadium, chromium, and iron. In one embodiment, the material forming outer tube 12 may be a superelastic material such as nitinol, which is a nickel-titanium alloy. In some embodiments, outer tube 12 may include layers of different materials and reinforcements. Outer tube 12 may have any cross-sectional shape and/or configuration and may be any desired dimension that can be received in a body cavity. In some embodiments, outer tube 12 may be made of, or coated with, a polymeric or lubricious material to enable endoscope 10 to pass through a body cavity with ease. Additionally, outer tube 12 may be steerable and may have areas of different flexibility or stiffness to promote steerability within the body cavity.

Outer tube 12 may include one or more channels 20. The one or more channels 20 may extend substantially longitudinally (axially) within outer tube 12, and generally between proximal end 10a and distal end 10b of endoscope 10. In particular, the one or more channels 20 may extend distally from handle portion 14 and terminate at distal end 12b of outer tube 12. The one or more channels 20 may have any suitable size, cross-sectional area, shape, and/or configuration to, for example, introduce medical instruments to distal end 10b of endoscope 10.

In the exemplary embodiment depicted in FIG. 1, outer tube 12 includes three channels 20. A medical instrument 24 is introduced through one of the three channels. The additional channels may introduce visualization devices (i.e., lighting sources and/or imaging sources) and treatment devices, such as a suction device, an injection needle, electrocautery needle, forceps, and any other suitable device known in the art to distal end 10b of endoscope 10. It should be understood, however, that outer tube 12 may include a greater or lesser number of channels. It is contemplated that additional channels may be provided for irrigation and/or aspiration.

Medical instrument 24 may be slidably inserted through a port 22 at proximal end 10a of endoscope 10 to enter channel 20. As shown in FIG. 1, port 22 is provided at an angle to channel 20 in outer tube 12. Medical instrument 24 may be advanced through channel 20, and a distal portion 24b of medical instrument 24 may be positioned distally of distal end 12b of outer tube 12. Distal portion 24b may be configured for use during a surgical method including diagnostic and/or therapeutic procedures. Specifically, distal portion 24b may be configured for use in dissection procedures such as, for example, endoscopic mucosal resection (EMR) and endoscopic submucosal dissection (ESD) procedures.

Distal portion 24b of medical instrument 24 includes a distal tool 28 fixed to distal end 26b of shaft 26. Distal tool 28 may be a cauterizing member configured to coagulate, cauterize, dissect, burn, and/or cut target tissue upon being energized by an electrical current. Distal tool 28 may be configured to perform monopolar or bipolar cauterization. Distal tool 28 may be formed of any material capable of conducting electricity, such as, for example, stainless steel, nickel titanium alloys, and the like, and may have any shape, size, and/or configuration. In the exemplary embodiment, distal tool 28 is a cautery tip having a substantially hemispherical shape.

Distal tool 28 is coupled to a wire 38 disposed in a first lumen 36 of shaft 26 of medical instrument 24 (FIG. 4) to provide an electrical pathway from a source of electricity (not shown) to distal tool 28. A handle at the proximal end of instrument 24 may include an appropriate connector for connection to, for example, a source of electrical energy. The energy may be conducted through the instrument handle to wire 38. Wire 38 may be formed of any material capable of conducting electricity, such as, for example, stainless steel, nickel titanium alloys, and the like. In some embodiments, distal tool 28 may be insulated from shaft 26 by insulation. In particular, portions of distal end 26b may be covered with a suitable insulating material, such as, for example, a powder coat or non-conducting polymeric sheath, to minimize the discharge and effects of any stray electrical energy from distal tool 28.

In alternative embodiments, a sharp distal tool such as, for example, a scalpel, a knife, scissors, or blades, or other electromechanical devices may be employed in place of the cauterizing distal tool 28 without departing from the scope of the disclosure. It is also contemplated that shaft 26 may form distal tool 28. In particular, a distal tip portion of shaft 26 may be uninsulated and connected to a source of cautery current to cauterize tissue.

Distal portion 24b of medical instrument 24 may further include an expandable member 30 positioned adjacent to distal end 26b of shaft 26, proximally of distal tool 28. The phrase “expandable member” generally relates to any expandable structure, such as a balloon or other inflatable structure, regardless of the elasticity of the material comprising the structure. For example, the phrase “expandable member” may denote a thin-walled structure made of material of low elasticity (which does not stretch significantly during inflation) or highly elastic material (which does stretch significantly during inflation). For example, expandable member 30 may be made from polyethylene terephthalate (PET), polyurethanes, polyethylenes and ionomers, copolyesters, rubbers, polyamides, silicone, latex, or any other suitable materials known in the art. Expandable member 30 may be configured for use as a blunt dissection tool.

FIG. 2 shows an exemplary embodiment of expandable member 30 in a collapsed configuration, and FIG. 3 shows an exemplary embodiment of expandable member 30 in an expanded configuration. As illustrated in FIGS. 2 and 3, expandable member 30 is disposed on shaft 26 such that, when expandable member 30 is in the collapsed configuration, expandable member 30 is folded about shaft 26, and when expandable member 30 is in the expanded configuration, expandable member 30 expands radially outward from shaft 26. In some embodiments, a portion of expandable member 30 may be received in shaft 26 so that medical instrument 26 presents a substantially constant cross-section along a length of shaft 26 when expandable member 30 is in the collapsed configuration.

Sometimes expandable members have a natural tendency toward roundness. In the present disclosure, the expanded exterior configuration of expandable member 30 has a substantially flat and wide shape in the expanded configuration. In particular, expandable member 30 may have a flat base 30a and a body 30b in the expanded configuration. In the exemplary embodiment illustrated in FIG. 3, base 30a and body 30b forming a wedge-shaped profile having an end 30e, an edge 30d, a substantially flat top 30c, and substantially flat side walls 30f. Base 30a and top 30c may meet at edge 30d, which may be straight and oriented perpendicular to the longitudinal axis of shaft 26. While in the exemplary embodiment, body 30b is tapers In a distal direction so that edge 30d is located closer to distal end 26b of shaft 26, it is contemplated that body 30b may be tapered towards the proximal end of instrument 24. In those embodiments, base 30a and top 30c may meet a proximal edge that is straight and oriented perpendicular to the longitudinal axis of the shaft 26. Expandable member 30 may be molded to have the wedge-shaped profile through a variation in wall thickness and pre-formed geometry, however, the wedge shaped profile may be formed by any other method known in the art.

The profile of expandable member 30 may provide certain benefits. For example, the substantially flat base 30a may rest on a tissue layer to be protected and body 30b of expandable member 30 may lift a tissue layer to be dissected away from the tissue layer to be protected as expandable member 30 expands from the collapsed configuration to the expanded configuration. In this manner, expandable member 30 may facilitate the separation of tissue layers during a dissection procedure. It is understood that expandable member 30 may alternatively have a flat base 30a and a body 30b that forms a dome, hemispherical, or rectangular profile, or any other shape known to one skilled in the art.

Referring to FIG. 4, expandable member 30 may be fluidly connected to a first fluid lumen 32 and a second fluid lumen 34 in shaft 26 of medical instrument 24. First fluid lumen 32 and second fluid lumen 34 may provide a fluid pathway through which a fluid, such as a liquid or gas, may pass to expand (inflate) and contract or collapse (deflate) the expandable member 30. In some embodiments, shaft 26 may include a single lumen to provide fluid to expandable member 30. The inflation fluid may be air, water, carbon dioxide, saline solution, or a contrast agent. In alternative embodiments, expandable member 30 may be mechanically, electrically, or pneumatically expanded and collapsed without departing from the scope of the disclosure.

A method of using medical instrument 26 will now be described. Once an endoscope 10 is provided at the treatment site, distal portion 24b of medical instrument 24 may be advanced through channel 20 of endoscope 10 to a desired tissue site 40. Distal portion 24b may be maneuvered to tissue site 40 so that distal tool 28 is positioned adjacent tissue site 40. Tissue site 40 may include two or more tissue layers. In the exemplary embodiment, tissue site 40 may include a first tissue layer 40a and a second tissue layer 40b. In some embodiments, the second tissue layer 40b may be a muscularis layer of an organ wall, and the first tissue layer 40a may be the mucosal and/or submucosal layer of an organ wall.

Once distal tool 28 is positioned adjacent tissue site 40, an electrical current may be supplied to distal tool 28. Distal tool 28 may be used to coagulate, cauterize, dissect, burn, and/or cut a small hole in first tissue layer 40a to gain access to a space A between first tissue layer 40a and second tissue layer 40b. Distal portion 24b, including expandable member 30, may then be inserted between tissue layers 40a and 40b through the hole in first layer 40a (FIG. 5A).

During insertion, expandable member 30 may be oriented relative to first tissue layer 40a and second tissue layer 40b so that, when expandable member 30 is expanded, base 30a will rest against a surface of second tissue layer 40b. Orientation may be achieved through endoscopic guidance and/or suitable markers on distal portion 24b of instrument 24. Fluid may then be delivered through one or both of first lumen 32 and second lumen 34 to expandable member 30 to inflate expandable member 30 from a collapsed configuration to an expanded configuration (FIG. 5B).

In an exemplary embodiment, expandable member 30 may have a wedge-shaped profile in the expanded configuration. The wedge-shaped profile may facilitate tissue separation by lifting the first tissue layer 40a away from the second tissue layer 40b as expandable member 30 expands from the collapsed configuration to the expanded configuration. In doing so, expandable member 30 may create an area B between tissue layer 40a and second tissue layer 40b.

In some embodiments, expandable member 30 may be deflated, repositioned, and inflated again to enlarge area B. The steps may be repeated until the desired area of tissue is separated. Medical instrument 26 may then be removed from area B and a separate treatment device may be introduced into area B to perform a dissection procedure. In alternative embodiments, expandable member 30 may be left in an expanded configuration to separate the first tissue layer 40a from the second tissue layer 40b. This may facilitate dissection of first tissue layer 40a by creating a “safety zone” between first tissue layer 40a and second tissue layer 40b. A separate treatment device may be introduced into area B between the first tissue layer 40a and second tissue layer 40b to dissect the portion of first tissue layer 40a containing, for example, a lesion. This may reduce the risk of perforation to second tissue layer 40b. Additionally and/or alternatively, expandable member 30 could remain in area B to lift a portion of first tissue layer 40a containing, for example, a lesion, and facilitate dissection of the portion of first tissue layer 40a with a separate treatment device such as, for example, a snare from outside of area B.

FIGS. 6 and 7 depict an exemplary distal portion 124b and the components thereof in accordance with a second embodiment of the disclosure. Distal portion 124b may be similar to distal portion 24b of the first embodiment. In particular, distal portion 124b includes an expandable member 130 configured to expand from a collapsed configuration (FIG. 6) to an expanded configuration (FIG. 7). As in the embodiment above, expandable member 130 has a base 130a and a body 130b that form a wedge-shaped profile having an end 130e, an edge 130d opposite end 130e, a substantially flat top 130c, and substantially flat side walls 130f in the expanded configuration.

Additionally, distal portion 124b includes a distal tool 128 fixed to distal end 126b of elongate shaft 126. In this embodiment, however, distal tool 128 is an injection needle. Injection needle is hollow and includes a fluid channel 128b having a distal opening. Fluid channel 128b may be fluidly coupled to a fluid source that can connect to a fluid port on the handle of medical instrument 24. The fluid port may be connected to channel 128b via a lumen in shaft 126. Distal tool 128 may additionally be formed of any material capable of conducting electricity, such as, for example, stainless steel, nickel titanium alloys, and the like. Distal tool 128 may be relatively blunt and penetrate tissue through an electrical current running through distal tool 128 so as to coagulate, cauterize, dissect, burn, and/or cut target tissue upon being energized by the electrical current.

Referring to FIGS. 8A-8C, a method of using medical instrument 126 will now be described. Once an endoscope 10 is provided at the treatment site, distal portion 124b of medical instrument 24 may be advanced through channel 20 of endoscope 10 to a desired tissue site 40. Distal portion 124b may be maneuvered to tissue site 40 so that distal tool 128 is positioned adjacent tissue site 40. Electrical current may be supplied to distal tool 128 to temporarily activate distal tool 128. Distal tool 128 may be used to provide an initial cut into tissue by coagulating, cauterizing, dissecting, burning, and/or cutting a small hole in first tissue layer 40a at tissue site 40 to gain access to a space A between first tissue layer 40a and second tissue layer 40b (FIG. 8A).

Distal portion 124b, including expandable member 130, may then be inserted into the small hole in the first layer of tissue 40a. During insertion, expandable member 130 may be oriented relative to first tissue layer 40a and second tissue layer 40b so that, when expandable member 30 is expanded, base 130a will rest against a surface of second tissue layer 40b. Orientation may be achieved through endoscopic guidance and/or suitable markers on distal portion 124b. Fluid may then be delivered through port 128b between first tissue layer 40a and second tissue layer 40b (FIG. 8B). In some embodiments, the fluid may assist in separating and identifying the tissue layers disposed between, for example, a lesion and the muscularis tissue.

After the tissue layers have been identified, inflation fluid may be delivered through one or both of first lumen 132 and second lumen 134 to expandable member 130 to inflate expandable member 130 from a collapsed configuration to an expanded configuration (FIG. 9C). In an exemplary embodiment, expandable member 130 may have a wedge-shaped profile in the expanded configuration. The wedge-shaped profile may facilitate tissue separation by lifting the first tissue layer 40a away from the second tissue layer 40b as expandable member 130 expands from the collapsed configuration to the expanded configuration. In doing so, expandable member 130 may create an area between tissue layers 40a and 40b.

In some embodiments, expandable member 130 may be deflated, repositioned, and inflated again to enlarge area B. The steps may be repeated until the desired area of tissue is separated. Medical instrument 126 may then be removed from area B and a separate treatment device may be introduced into area B to perform a dissection procedure. In alternative embodiments, the expandable member 130 may be left in an expanded configuration to separate first tissue layer 40a relative to second tissue layer 40b. This may facilitate dissection of first tissue layer 40a by creating a “safety zone” between first tissue layer 40a and second tissue layer 40b. A separate treatment device may be introduced area B between the first tissue layer 40a and second tissue layer 40b to dissect the portion of first tissue layer 40a containing, for example, a lesion. This may reduce the risk of perforation to second tissue layer 40b. Additionally and/or alternatively, expandable member 130 could remain in area B to lift a portion of first tissue layer 40a containing, for example, a lesion, and facilitate dissection of the portion of first tissue layer 40a with a separate treatment device such as, for example, a snare from outside of area B.

Alternative non-limiting examples of expandable member having a flat base in an expanded configuration are shown in FIGS. 9 and 10. In FIG. 9, expandable member 230 may include a series of inflation chamber 232. Inflation chambers 232 may be arranged so as to provide a substantially flat base in the expanded configuration. Inflation chambers 232 may be inflated simultaneously through a small hole between chambers, or inflated independently through holes in the instrument shaft.

In FIG. 10, expandable member 330 may be molded with strand structures 332 to create the desired shape and/or configuration. Strand structures 332 may be formed of flexible material such as, for example, elastic or rubber which may expand radially when expandable member 330 expands from the collapsed configuration to the inflated configuration. Strand structures 332 may be configured to have a relaxed arrangement when expandable member is in a collapsed configuration, and may be configured to restrain the inflation of portions of expandable member 330, when expandable member 330 expands from the collapsed configuration to the expanded configuration. In this manner, portions of expandable member 330 that do not have strand structures 332 may inflate naturally and portions of expandable member 330 having the strand structures 332 may be restrained to shape expandable member 330 to have the desired profile and/or configuration.

Other inflation patterns and shapes and/or configurations of the expandable member are also contemplated. For example, the expandable member may inflate in a spiral path, radially outward path, in concentric rings, and/or in an expanding grid pattern from the collapsed configuration to the expanded configuration. In the expanded configuration, the expandable member may form a dome, tapered, square, rectangular, triangular, or cross-like profile, or any other profile known to those skilled in the art. Additionally and/or alternatively, the expandable member may have varying thicknesses across the length of the expandable member in the expanded configuration. For example, the expandable member may have a body that forms a dome profile with thin edges or, alternatively, the expandable member may have rounded edges with a substantially thin or flat body profile.

Another exemplary embodiment of the present disclosure may include a blunt separation device disposed at the distal end of an introduction sheath, such as the endoscope 10. The device may include a hollow elongate member (which may be configured as a cap in some embodiments) having a blunt distal tip configured with a cautery element. The elongate member can be formed from transparent material, so that the cap is entirely transparent, or a portion of the cap may be transparent, in the nature of a window. A visualization mechanism, such as a camera, may be positioned within the transparent elongate member to view the surrounding cavity. The cautery element may be configured to form a small hole in a tissue layer, such as, e.g., the mucosa, allowing the blunt tip to move forward into the tissue, forcing adjacent layers apart. The operator employs the visualization mechanism to facilitate the process. Advancing the cap performs blunt separation along the interface between two layers.

In an embodiment, the blunt distal tip may be atraumatic to avoid tissue damage. In addition, an expandable member may be disposed at the outer surface of the cap, and as noted in connection with the cap, the expandable member may be completely or partially transparent. The expandable member remains in a collapsed state during the device insertion, and once the device has moved completely into the tissue, the expandable member may be expanded, which has the effect of further forcing the adjacent tissue layers apart, allowing the cap to be moved further into the tissue. These operations are reiterated until the operator has separated the desired amount of tissue. Further, if desired, the separated tissue may be resected or dissected, either by a separate device or by a device deployed from the same endoscopic device. Other actions, such as tissue retrieval, may also be performed as desired.

In the following sections, embodiments of the present disclosure will be described using an exemplary body organ—the esophagus. The embodiments of the medical device discussed below aim to remove a lesion on the mucosal layer of the esophagus without damaging the underlying muscularis layer. It will be understood, however, that the esophagus is merely exemplary and that the device may be utilized in other suitable organs, such as, the stomach, colon, duodenum, or any other organ that may require tissue resection. Further, tissue resection is not limited to removal of lesions. Any desired target tissue may be resected in accordance with the principles of the present disclosure. Further, although the principles of the present disclosure are described in connection with the mucosal and muscularis tissue layers, those of ordinary skill in the art will recognize that the principles of the present disclosure may be used to separate any two tissue layers.

FIGS. 11A and 11B illustrate an exemplary embodiment of a medical device 400 configured to separating tissue layers along natural interfaces in collapsed and expanded states, respectively. As shown, the medical device 400 includes an introduction sheath 402 having a proximal end 404, a distal end 406, and a lumen extending between the proximal and distal ends 404, 406. The distal end 406 may be coupled to a cap 408, which in turn may include one or more expandable members, such as balloons 410A-B. A tip 412 lies at the distal end of the device 400, and that element may be formed to facilitate entry into the target tissue or to facilitate the blunt separation function. Depending on the desired primary function, as known in the art, tip 412 may have a relatively sharp point (promoting penetration) or a rounded or beveled structure (promoting tissue separation). Other shapes may be chosen as desired. In the exemplary embodiments, the tip 412 may present an atraumatic tip having any known structure suitable for avoiding tissue damage while accomplishing the process goals described above.

Introduction sheath 402 may be endoscope 10 or another suitable introduction device or sheath adapted to be moved into a body lumen. In the illustrated embodiment, introduction sheath 402 may include one or more channels 414, through which the operator may insert a visualization mechanism 415, which may include a light source and an imaging means, such as, e.g., a camera. Alternatively, the visualization mechanism 415 may be any other imaging mechanism useful for allowing the operator to the surgical site, such as, ultrasound or infrared sensors or the like, disposed at the distal end 406.

The introduction sheath 402 may be a tubular structure. This structure may have a substantially circular cross-section or an elliptical, oval, polygonal, or irregular cross-section may be employed, as desired. In addition, a select portion of the introduction sheath 402, such as, e.g., a distal portion, may have cross-sectional configuration or dimension different from another portion, e.g., a proximal portion, of introduction sheath 402. Moreover, the introduction sheath 402 may be flexible along its entire length or adapted for flexure along portions of its length. Alternatively, the distal end 406 may be flexible while the remainder of the introduction sheath 402 may be rigid. Flexibility allows the introduction sheath 402 to maneuver turns in body lumens, while rigidity provides a structure upon which the operator can exert the necessary force to urge the introduction sheath 402 forward. As known in the art, introduction sheath 402 may be fitted with steering capability, actuated by control lines or rods. Steering devices are well known in the art and will not be described further here.

The diameter of the introduction sheath 402 may be selected based on the desired application, with the largest diameter generally chosen to be smaller than the typical diameter of the desired body lumen where the introduction sheath 402 may be used. A sheath 402 to be employed in the esophagus, for example, will generally be smaller than a sheath 402 to be employed in the colon. Similarly, the length of the introduction sheath 402 may vary according to the location of the body lumen where the tissue separation is to be conducted.

Introduction sheath 402 may be made of any suitable biocompatible material such as a polymeric, metallic, or rubber material. The introduction sheath 402, or a portion thereof, may be also made from a malleable material, such as stainless steel or aluminum, allowing a physician to change the shape of the introduction sheath 402 before or during an operation. In some instances, the introduction sheath 402 may be composed of an extrusion of wire braided polymer material to impart flexibility. The introduction sheath 402 may also be coated using suitable low friction material, such as TEFLON®, polyetheretherketone (PEEK), polyimide, nylon, polyethylene, or other lubricious polymer coatings, to reduce surface friction with the surrounding body tissues.

In general, the introduction sheath 402 may be any known endoscopic device used for colonoscopy, resectoscopy, cholangioscopy, or mucosal resection. Such devices are well known in the art, and thus introduction sheath 402 will not be discussed in further detail.

Cap 408 may be a relatively short, generally hollow member adapted to fit over the distal end 406. The shape of cap 408, as well as the material from which it is formed, are selected provide for blunt separation of tissue layers in a selected bodily cavity. In general, the cap 408 may be an elongate, tubular member, closed at its distal end 420 and open at its proximal end 418, with an interior portion 422. A blunt or atraumatic tip 412 may extend from the cap's distal end 420, and one or more expandable members, such as balloons 410A-B, may be disposed on the outer surface of the cap 408. Proximal end 418 may be shaped to fit over the distal end 406 of the appropriate introduction sheath 402, for which shape of the proximal end 418 may be circular, though other shapes may be employed. In addition, the cross-sectional dimensions of the cap 408 may be uniform or may vary along its length, as seen in the taper profile of the illustrated embodiment.

The dimensions of the cap 408 may vary according to the desired application of the medical device 400. For example, if medical device 400 is to be inserted through the urethra of a patient, the diameter of the cap 408 may be considerably smaller than a similar device used in connection with colonoscopy.

In addition, cap 408 may be configured to facilitate visualization of the tissue. In some embodiments, that feature can be achieved by providing the entirety or a portion of the cap 408 as a transparent material. Materials suitable for that task are set out in detail below. Some embodiments may provide the entire cap 408 as being transparent, while others may provide only a portion in that condition, or others may provide a transparent window. Any of these alternatives may allow an operator to view tissue around the cap 408 by way of the visualization mechanism 415. As discussed the visualizing mechanism 415 may be introduced within the cap 408 through one of the channels 414 of the introduction sheath 402, or the visualization mechanism 415 may be affixed within interior portion 422 of the cap 408 to visualize the surrounding cavity. The illustrated embodiment of the present disclosure uses a completely transparent cap 408 to facilitate visualizing the surrounding tissue.

The cap 408 may include openings 424. The openings 424 may allow any medical tools present within the channels 414 to communicate with the surrounding body cavity. In some embodiments, some of the openings 424 may be completely separate from the one or more expandable balloons 410A-B, so that devices extending outward through the openings 424 do not interfere with the expansion of the expandable balloons 410A-B. In addition, the openings 424 may further aid in removal of resected tissue from the body cavity through the channels 414 of the introduction sheath 402. In addition, in some embodiments, some of the openings 424 may assist in expansion of the expandable balloons 410A-B by providing a connection means between the channels 414 and the expandable balloons 410A-B. Further, in some embodiments, one or more openings 424 may connect to the tip 412 to provide cutting tools or devices to the tip 412.

The cap 408 may be detachably connected, permanently coupled, or formed as an integral component of the introduction sheath 402. Cap 408 may be coupled to distal end 406 by any suitable coupling mechanism, such as assemblies joined by snap fit, a screw fit, a luer-lock, key/slot, or other known attachment mechanisms. Suitable permanent coupling methods may include gluing or spot welding, depending on the cap 408 material. Cap 408 may be also be introduced through a working channel 414 of the introduction sheath 402 and the depth of the cap 408 extending from the distal end 406 may be adjustable. In such embodiments, an airtight seal may be maintained between cap 408 and introduction sheath 402. Alternatively, cap 408 may be formed integral with the distal end 406 of the introduction sheath 402.

Materials suitable to fabricate the cap 408 include those capable of providing biocompatibility, together with at least some degree of rigidity. Either all or portions should be transparent. Further, a biocompatible material providing lubricity or delivery of desired compounds, e.g., drugs or other therapeutic agents, to the patient may be coated over the outer surface of cap 408.

An atraumatic tip 412 may extend distally from the cap 408. This element may be generally blunt in form and may be carried on the cap 408 at or near the longitudinal axis. The atraumatic tip 412 may be configured to perform blunt separation of targeted tissue. The atraumatic feature may be achieved with beveled or rounded ends, for example. The atraumatic tip 412 may prevent inadvertent damage to tissue during maneuvers within a body lumen. Further, the atraumatic tip 412 may assist in placement of the medical device 400 between tissue planes as noted below.

The dimensions and characteristics of the atraumatic tip 412 may vary based on its application and intended use. Given its role in blunt separation, the atraumatic tip 412 may present some degree of rigidity, depending on the separation scenario. In one embodiment of the present disclosure, flexibility may vary longitudinally such that the distal end of the tip 412 may be more flexible than its proximal end. In some embodiments, the tip 412 may taper distally, while in other embodiments the tip 412 may be rounded. Other embodiments may call for a tip 412 that is relatively thinner in the middle and relatively thicker at its proximal and distal ends. Such atraumatic design features may also facilitate insertion and moving the medical device 400 within a patient's body.

The tip 412 may include mechanisms such as motors, strings or other actuators to allow an operator to steer the tip 412 within a body cavity. The steerability of the tip 412 may be independent of the steerability of the introduction sheath 402. For example, the operator may be able to steer or position the tip 412 in a same or different orientation relative to the remainder of the cap 408.

The atraumatic tip 412 may be integral to the cap 408 or an external element attached to the cap 408 using any suitable attachment methods. For example, adhesives, such as, biocompatible resins, or glue may be used to attach the tip 412 to the distal end 420. Other attachment methods may include use of wire connections, heat welding, or mechanical joints. As alluded to above, tip 412 may be fabricated from a one-piece construction with cap 408.

The atraumatic tip 412 may be fabricated from any biocompatible polymeric, rubber, or metallic material. For instance, rigid or semi-rigid materials, such as, e.g., metals (including shape-memory materials such as Nitinol), super elastic materials, polymers, resins, or plastics may be used. Atraumatic tip 412 may also be optically transparent, allowing the physician to view the targeted tissue disposed within the body cavity. Further, a biocompatible lubricating material may be applied as a coating over the outer surface of atraumatic tip 412.

In addition, the atraumatic tip 412 may include a cutting tool 426. The cutting tool 426 may be any suitable tool, such as, a surgical blade, a snare loop, a laser fiber, a cautery tool or the like. An operator may use the cutting tool 426 to cut tissue at a desired location within the body cavity proximate the targeted tissue. The cutting tool 426 may be operatively positioned at the distal end of the atraumatic tip 412. The cutting tool 426 may be permanently coupled to the atraumatic tip 412 or it may be slidably disposed within one of the channels 414 with the resection tool extending out of the atraumatic tip 412 through an opening 424.

In some embodiments of the present disclosure, the cutting tool 426 may include mechanical (surgical blades), thermal, electro, or chemical cautery. For example, in the illustrated embodiment, the cutting tool 426 may be configured as an electrocautery tool. The cutting tool 426 may be configured as an electrical contact made of a conductor material, such as, copper, silver, or gold wire, or it may be a heating element, such as, e.g., tungsten. It may occupy all or a portion of the atraumatic tip 412. The cutting tool 426 may be connected to an electrocautery system (not shown) through suitable connections e.g., wires, extending through one or more channels 414, and it may be switchable between cutting and coagulation.

Alternatively, in some embodiments, the tip 412 may not be atraumatic, but may include a percutaneous tip. In some embodiments, this tip 412 may include a cutting tool such as a needle or a surgical blade. Further, in such embodiments, the tip 412 may be covered by any actuatable atraumatic element (not shown) that may be removed selectively using any actuation means. In covered state of the tip 412, atraumatic element may assist the operator to maneuver the medical device 400 within a body cavity without causing any inadvertent damage to the contacting tissues. Further, when the atraumatic element is removed upon actuation, the tip 412 may be used to cut tissue.

As mentioned above, one or more expandable members (shown as expandable balloons 410A-B) may be disposed on the outer surface of cap 408. These devices expand upon actuation and thus may include expandable elements such as balloons, cages, linkages, foam, baskets, or the like. As the expandable members expand, they may serve to separate adjacent tissue layers, bluntly separating them. Although the illustrated embodiment depicts two expandable balloons 410A-B as the expandable members, those of ordinary skill in the art will readily recognize that cap 408 may include any numbers of expandable elements functioning as the expandable members. Further, in embodiments having two or more expandable members, each expandable member may be of a different type, form, and/or configuration that the other expandable members.

As discussed, in the illustrated embodiment of FIGS. 11A and 11B, the expandable balloons 410A-B function as the expandable members. The expandable balloons 410A-B may be placed diametrically opposed to each other about the longitudinal axis of the cap 408. The expandable balloons 410A-B may be transparent to aid in visualization of the surrounding cavity. In some embodiments, the expandable balloons 410A-B may be entirely transparent while in some other embodiments a portion of the expandable balloons 410A-B may be transparent to facilitate visualization using the visualization mechanism 415. Other designs may provide opaque or semitransparent expandable balloons 410A-B, depending on other features to facilitate viewing. For example, one or more portions of each of expandable balloons 410A-B may facilitate viewing tissue disposed in the surrounding body cavity.

Before operation of the medical device 400, the expandable balloons 410A-B may be carried in a collapsed configuration around the cap 408 as illustrated in FIG. 11A. Alternatively, the expandable balloons 410A-B may be disposed within one or more channels 414 in collapsed configuration, and may extend out from the channels 414 through openings 424 to expand. Further, cap 408 may include one more recesses or other cavities/openings disposed along a side portion for holding expandable balloons 410A-B in a collapsed configuration, such that the expandable balloons 410A-B are maintained substantially within an outer periphery of cap 408.

The expandable balloons 410A-B may be operatively coupled to a suitable expansion mechanism. That mechanism may expand the expandable balloons 410A-B to an expanded configuration by any suitable means known in the art. In the exemplary embodiment having expandable balloons 410A-B, the expansion mechanism may inflate the balloons 410A-B by using an inflation fluid to or any other suitable expansion method. The inflation fluid may be filled within the balloons 410A-B to expand them into the expanded configuration and expelled from them to bring them to a collapsed configuration. The inflation fluid may be air, gas, saline, or any other biocompatible fluid. In such embodiments, the expansion mechanism may include conduits 428 that may provide inflation fluid to the balloons 410A-B, via, e.g., channels 414 and openings 424.

In addition, the expansion mechanism in such embodiments may include a pressure source (not shown), a controller (not shown), and a fluid storage device (not shown), operatively connected to the conduits 428. The pressure source may be any pressurizing device, such as a mechanical pump, electrical pump, a syringe, or the like, and the fluid storage device may be a fluid cylinder, tank, or similar device. The pressure source may apply pressure to inflate or deflate the balloons 410A-B by transferring fluid from the fluid storage device to or from the balloons 410A-B. The controller (not shown) may control the operation of the pressure source by activating or deactivating the pressure source, or controlling the flow rate and volume.

FIG. 11B illustrates the expandable balloons 410A-B in their expanded state. The inflated dimensions of balloons 410A-B may be designed for particular operating scenarios. For example, for separation of tissue layers in the small intestine, the balloons 410A-B may be considerably smaller, while larger sizes will be more suitable in organs such as the stomach. The balloons 410A-B may assume any suitable structure such as spherical, cylindrical, or conical as desired. For example, in some embodiments, the balloons 410A-B may have a tapered leading edge structure, similar to an unswept wing. In such embodiments, the width of a balloon 410A-B may increase from its edge towards the point where the balloon 410A-B connects the cap 408. Similarly, the dimensions of the balloons 410A-B may depend upon the diameter of the body cavity in which the medical device 400 may be used. Further, the balloons 410A-B may be coupled to the cap 408 by a suitable coupling mechanism (not shown), such as a mechanical attachment (e.g., a hook) or an adhesive.

The balloons 410A-B may be formed from any suitable transparent, waterproof, fire resistant, biocompatible, and elastomeric material. Polymeric, rubber, or other material possessing such properties can be employed. Those in the art are well aware of the range of suitable and available materials.

The outer surface of the cap 408 may include indicia, visible under various imaging regimes. For example, radiopaque or sonoreflective markings (not shown) may be added to an exterior surface of the cap 408 to indicate position and orientation of the cap 408 during a procedure. That information can enable the surgeon to track the medical device 400 and avoid potential damage to sensitive tissues.

Moreover, to inhibit bacterial growth in the body cavity or in the mucosal wall, cap 408 may be coated with an antibacterial coating (not shown). The coating may contain an inorganic antibiotic agent, disposed in, e.g., a polymeric matrix, which may aid the antibiotic agent to adhere to the cap 408 surface. Other suitable coatings may also be applied to one or more surfaces of cap 408 and/or expandable balloons 410A-B.

FIG. 12 illustrates another embodiment 200 of the medical device 400 including a single expandable member, such as a balloon 510. The balloon 510 may have similar dimensions, material, expansion mechanism, and coupling mechanism as illustrated with the embodiments described in FIGS. 11A and 11B. In addition, in this embodiment, the expansion mechanism may utilize a single conduit 528 for expansion of the balloon 510.

In an alternative embodiment, the expandable member(s) may be configured as radially expanding basket(s) (not shown), as noted above. The basket may assume two configurations—expanded and collapsed. The basket may remain collapsed within a channel 414 (or any other suitable cavity or opening disposed in a side of cap 408) during insertion and retrieval of the medical device 400, or the basket may lie on the outer surface of the cap 408. Once deployed, the basket may extend from the distal end 406 of the channel 414 and expand in a radial direction. In the expanded state, the basket may perform blunt separation by pushing into the surrounding tissue, thereby separating adjacent tissue layers. The basket may be a wire mesh made of a shape memory alloy, such as nitinol. In some embodiments, the basket may be configured as a stent. In such embodiments, the stent may be self-expandable, or may be expanded with the aid of a suitable expansion mechanism, such as a spring mechanism (not show), or it may expand as the shape memory alloy returns to its original configuration.

FIG. 13 exhibits another alternative embodiment of the medical device 400. In this embodiment, the medical device 400 may contain a reciprocation mechanism to move the cap 408 longitudinally back and forth, as indicated by the phantom image in FIG. 13. The reciprocation may facilitate driving the cap 408 between tissue layers within a body cavity, in a manner similar to that of a jackhammer. A variety of techniques may be used to impart reciprocation to the cap 408. In the illustrated embodiment, a semi-rigid center core 602 may be operationally connected to the cap 408 within the introduction sheath 402. The semi-rigid center core 602 may be reciprocated, in turn moving the cap 408 and/or tip 412. In some embodiments, the position 604 of the tip 412 and cap 408 may represent the maximum position of tip 412 and cap 408 within a stroke. The reciprocation of tip 412 and/or the cap 408 may have a stroke of suitable length, which may be in the range of 0 to 0.25 inches. Alternatively, any actuation device, working through control means extending through the introduction sheath 402, may be connected to the cap 408 to generate the reciprocal motion, at a suitable frequency chosen to may drive the cap 408 and/or tip 412 between desired tissue layers.

FIGS. 14A and 14B illustrate an exemplary method of using the medical device 700. The embodiments of the present disclosure may be employed to perform EMR of gastrointestinal, colonic, and esophageal cancer, small polyps, or cancerous masses that form along the mucosa and often extend into the lumens of the organs. In addition, those of ordinary skill will recognize that the principles of the present disclosure may be used to separate tissue layers in any suitable body location. The present disclosure allows for convenient resection of target tissue on the mucosal layer 40A while minimizing the risk of perforating the muscularis layer 40B.

Referring to FIG. 14A, an operator may insert the medical device 700 into a lumen of a patient's gastrointestinal tract 700, e.g., gaining entry by a natural orifice or by a small incision. The operator may introduce the visualization mechanism 415 to assist in maneuvering the medical device 700 to the surgical site. Once at a desired site, the operator may use the visualization mechanism 415 to examine the mucosal layer 40A and determine, e.g., whether the observed condition requires resection.

If resection is required, the operator may position the medical device 700 at a point near the lesion, positioned to allow resection of sufficient tissue around the lesion to ensure that all undesirable tissue is removed. To accomplish that task, the operator will dissect a flap of tissue including the complete lesion.

In the pre-insertion state of the medical device 700, the expandable balloons 410A-B may be in a collapsed state, folded around or partially inside the cap 408. The operator may now use the cutting tool 426 to cut a small incision in the mucosal layer 40A, sufficiently large to allow insertion of the atraumatic tip 412. The operator may then insert the cap 408, leading with atraumatic tip 412, between the mucosal layer 40A and muscularis layer 40B by thrusting it between the layers manually, separating the layers along their layer boundaries. Visualization mechanism 415, viewing through a transparent portion of cap 408, allows the operator to exercise direct observation and control of this process. Alternatively, the oscillation mechanism described above may assist in advancing cap 408.

Referring to FIG. 14B, after positioning the cap 408 sufficiently between the layers, the operator may expand the expandable balloons 410A-B, which may exert forces on the adjacent tissue layers, gradually separating them. As the expandable balloons 410A-B expands, the cap 408 may be able to be moved further into the gap.

Once the expandable balloons 410A-B have exerted their maximum effect in a given position, the operator may collapse them. The operator may reiterate the process, manually advancing cap 408 and pressing the layers apart. The manual moving forward/expansion cycle can be repeated until the entire targeted tissue layer is separated from any underlying layer.

When the desired area of mucosal layer 40A tissue is separated from the muscularis layer 40B, e.g., the operator may resect the separated mucosal layer 40A or desired portions thereof. For that purpose, it may be most desirable to leave the medical device 700 in position below the dissected tissue, and to resect that tissue employing a second medical device 700. The medical device 700 left below the dissected tissue may act as a shield between the muscularis layer 40B and the second medical device 700, preventing any inadvertent damage to the muscularis layer 40B during the resection. Alternatively, in some embodiments, a resection tool could be deployed from the medical device 700, or the operator could employ cutting tool 426. In such embodiments, the medical device 700 may act as a buffer between the dissected tissue layers and may create a cavity between the mucosal and muscularis layers 40A, 404. The resection tool or cutting tool 426 may resect the mucosal layer 40A within the cavity without contacting the muscularis layer 40B thereby preventing any damage to the muscularis layer 40B.

Embodiments of the present disclosure may be used in any medical procedure, including any medical procedure where resection of a layer of targeted tissue is required without causing harm to the underlying tissue layers. In addition, at least certain aspects of the above-mentioned embodiments may be combined with other aspects of the embodiments, or removed, without departing from the scope of the disclosure.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A medical instrument comprising:
a shaft having a distal end configured to cauterize tissue; and
an expandable member configured for insertion between a first tissue layer and a second tissue layer adjacent the first tissue layer, wherein the expandable member is configured to expand from a collapsed state to an expanded state to separate the first tissue layer from the second tissue layer, and wherein at least a portion of the expandable member has a substantially flat base in the expanded state.

2. The medical instrument of claim 1, wherein a body of the expandable member has a substantially tapered shape in the expanded state.

3. The medical instrument of claim 1, wherein at least a portion of the expandable member has a wedge-shaped profile in the expanded state.

4. The medical instrument of claim 1, wherein the shaft has a cautery tip.

5. The medical instrument of claim 1, wherein the expandable member includes a plurality of inflation chambers.

6. The medical instrument of claim 1, wherein the expandable member includes a substantially flat base and a body having a substantially flat top surface, wherein the substantially flat top surface and the substantially flat base meet at an edge that is straight and oriented perpendicular to a longitudinal axis of the shaft.

7. The medical instrument of claim 1, further including a channel extending through the shaft to a distal opening, wherein the channel is configured to deliver fluid.

8. A method of separating tissue layer, comprising:
positioning a distal portion of a medical instrument, including a shaft having a distal end configured to cauterize tissue, and an expandable member adjacent the distal end of the shaft, adjacent a tissue site;
inserting the distal portion between a first tissue layer and a second tissue layer at the tissue site; and
expanding the expandable member from a collapsed configuration to an expanded configuration to separate the first tissue layer from the second tissue layer, wherein at least a portion of the expandable member has a wedge-shaped profile in the expanded configuration.

9. The method of claim 8, further including:
collapsing the expandable member;
positioning the expandable member at a second location of the tissue site; and
expanding the expandable member to separate the first tissue layer from the second tissue layer.

10. The method of claim 8, further including, prior to expanding the expandable member, injecting fluid between the first tissue layer and the second tissue layer.

11. The method of claim 8, further including, after expanding the expandable member, inserting a treatment device between the first tissue layer and the second tissue layer to dissect a portion of the first tissue layer.

12. The method of claim 1, wherein the first tissue layer is the mucosal layer, and the second tissue layer is the muscularis layer.

13. The method of claim 8, further including, prior to inserting the distal portion between the first tissue layer and the second tissue layer, cutting the first tissue layer with the distal end of the instrument.

14. A tool comprising:
a cap member having a proximal end and a distal end, wherein the proximal end is configured to be secured to a distal portion of an introduction sheath, and wherein at least one of the cap member or a portion of the cap member is transparent;
a tip extending distally from the distal end of the cap member; and
at least one expandable member secured to the cap member, wherein the expandable member is configured to transition between a collapsed state and an expanded state.

15. The tool of claim 14, wherein the tip is configured to cauterize tissue.

16. The tool of claim 14, wherein the tip includes an atraumatic configuration.

17. The tool of claim 14, wherein at least one of the at least one expandable member or a portion of the at least one expandable member is transparent.

18. The tool of claim 14, wherein the tip is configured to move independently of the cap member.

19. The tool of claim 14, further including a reciprocation mechanism for longitudinally reciprocating the cap member.

20. The tool of claim 19, wherein the reciprocation mechanism extends through the introduction sheath and operatively connects to the cap member, and drives reciprocation of the cap member longitudinally relative to the introduction sheath.
